(19) United States
(12) Patent Application Publication
(10) Pub. No.: US 2021/0115367 A1
(43) Pub. Date: Apr. 22, 2021

(54) Use of Resorufin for Monitoring Metabolic Activity of Cells under Anaerobic Condition

(71) Applicant: General Automation Lab Technologies Inc., San Carlos, CA (US)

(72) Inventors: Jude Dunne, San Carlos, CA (US); Talia Jewell, San Carlos, CA (US); Alexander Hallock, San Carlos, CA (US)

(21) Appl. No.: 17/073,361

(22) Filed: Oct. 18, 2020

Related U.S. Application Data
(60) Provisional application No. 62/923,321, filed on Oct. 18, 2019.
(60) Provisional application No. 63/009,398, filed on Apr. 13, 2020.

(51) Int. Cl. C12M 3/00; C12M 1/00; C12M 1/06; C12M 1/42; C12M 1/34

(52) U.S. Cl. CPC C12M 21/08; C12M 23/20; C12M 23/34; C12M 41/40; C12M 27/02; C12M 35/04; C12M 35/08; C12M 23/38

(57) ABSTRACT

A method for identifying a status of at least one cell in a cell culture including resorufin in an anaerobic atmosphere is provided. In the method, the extent of reduction of resorufin in the cell culture to dihyrdoresorufin is measured while the cell culture is maintained in an anaerobic atmosphere. The cell culture may be loaded in microwells of a microfabricated chip positioned in an anaerobic chamber, and the measurement can be based on fluorescence of the cell culture.

### CROSS REFERENCE TO RELATED APPLICATIONS

This application claims priority to U.S. Provisional Application No. 63/009,398 filed Apr. 13, 2020, and to U.S. Provisional Application No. 62/923,321, filed Oct. 18, 2019, the disclosure of each of which is incorporated by reference herein in its entirety.

### BACKGROUND

Determination of cell viability, metabolic activity, and/or cell proliferation is important in a wide range applications.

Resazurin (7-Hydroxy-3H-phenoxazin-3-one 10-oxide) is a blue dye, itself weakly fluorescent until it is irreversibly reduced to the pink colored and highly fluorescent resorufin. A reducing environment correlates strongly with cell growth and resazurin is known to be nontoxic, so its use is common in animal cells, bacteria, and fungi for cell culture assays such as cell counting, cell survival, and cell proliferation.

The reduction of resazurin is shown in the below schematics:

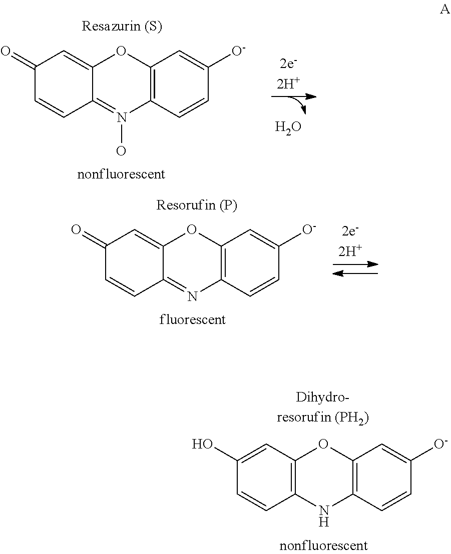

In aerobic (oxidizing) conditions, resazurin starts in an oxidized state and is reduced to resorufin by cell growth or proliferation. Therefore, monitoring the change of resazurin (i.e., color and/or fluorescence) can be used to indicate cell growth or proliferation in aerobic conditions. In an anerobic environment, however, resazurin cannot be used in the same way because the environment itself reduces the molecule, so one cannot distinguish where cell growth/proliferation is or is not occurring.

The most common methods for indicating cell metabolic activity in an anerobic chamber are (1) colorimetric indicators and (2) optical density. However, because of their low sensitivities, such methods have difficulties in producing reliable results in reaction chambers of minute volumes.

### SUMMARY OF THE INVENTION

In one aspect, the present disclosure provides a method for identifying a status of at least one cell in a cell culture including resorufin in an anaerobic atmosphere. In the method, the extent of reduction of resorufin in the cell culture to dihyrdoresorufin is measured while the cell culture is maintained in an anaerobic atmosphere.

In some embodiments, the measuring comprises measuring the fluorescence of the cell culture.

In some embodiments, the at least one cell can be loaded in one or more microwells of a microfabricated chip. In some embodiments, the at least one cell can be loaded in one or more droplets on a droplet-based platform.

The at least one cell can comprise a prokaryotic cell, a eukaryotic cell, or a bacterial cell.

In some embodiments, the measuring is performed for a plurality of times.

In some embodiments, the cell culture can be prepared by first mixing resorufin with a culture media, and then combining the at least one cell with the resorufin-loaded culture media.

In some embodiments, the status of the at least one cell can comprise the metabolic activity of the at least one cell.

In some embodiments, the method further includes: determining the presence or absence of at least one biological entity in the cell culture based on the measured extent of reduction of resorufin to dihydroresorufin.

In another aspect, a method of using a high density cell cultivation platform comprising a plurality of experimental units, is provided. The method includes: loading a sample onto the high density cell cultivation platform such that at least one experimental unit of the plurality of experimental units includes at least one cell, an amount of a nutrient, and resorufin; culturing a plurality of cells from the at least one cell in the at least one experimental unit in an anaerobic atmosphere; and measuring fluorescence of the contents of the at least one experimental unit. In some embodiments, the high density cell cultivation platform is a microfabricated device having a top surface defining an array of microwells as experimental units, the microwells having a surface density of at least 500 microwells per cm² or at least 750 microwells per cm². In some embodiments, the microwells each have a volume of no more than 5 nL. In some embodiments, the method further includes: determining the presence or absence of at least one biological entity in the at least one experimental unit based on the measured fluorescence. In some embodiments, the method further includes: based on the measured fluorescence, determining whether to select and transfer some cells from the plurality of cells to one or more target location.

In some embodiments, the high density cell cultivation platform is a droplet-based platform and the plurality of experimental units each comprise to a droplet on the droplet-based platform.

### BRIEF DESCRIPTION OF THE DRAWINGS

FIG. 1 is a perspective view illustrating a microfabricated device or chip in accordance with some embodiments.

FIGS. 2A-2C are top, side, and end views, respectively, illustrating dimensions of microfabricated device or chip in accordance with some embodiments.

FIGS. 3A and 3B are exploded and top views, respectively, illustrating a microfabricated device or chip in accordance with some embodiments.

FIG. 4A shows images of a microfabricated chip with array of microwells at 0, 15, and 39 hours of cultivation; FIG. 4B shows fluorescent signal intensities of the wells at 0 hours vs 15 hours; FIG. 4C show fluorescent signal intensities of the wells at 0 hours vs 39 hours.

FIG. 5 is a bar graph showing isolates at species level recovered from certain fecal samples cultured on a microfabricated chip platform using an embodiment of the method of the present invention.

FIG. 6 is a bar graph showing relative population at genus level of strains recovered from certain fecal samples cultured on a microfabricated chip platform using an embodiment of the method of the present invention with different media.

### DETAILED DESCRIPTION OF THE INVENTION

The present invention relates, in part, to cell viability, metabolic activity, cell proliferation, and cytotoxicity assays, especially suitable for use with high throughput devices.

One object of the disclosed subject matter is to provide a method to analyze and/or screen cells in an anaerobic condition or atmosphere based on cell growth, metabolic activity, and/or viability using resorufin.

In some embodiments, the methods of the present disclosure are practiced on a high density cell cultivation platform. The cultivation platform can be a highly partitioned system which comprises a high density array or arrays of microscale experimental units, where each microscale experimental unit can accommodate one or more cells and provide an environment independent and separate from other microscale experimental units for cell cultivation, growth and proliferation.

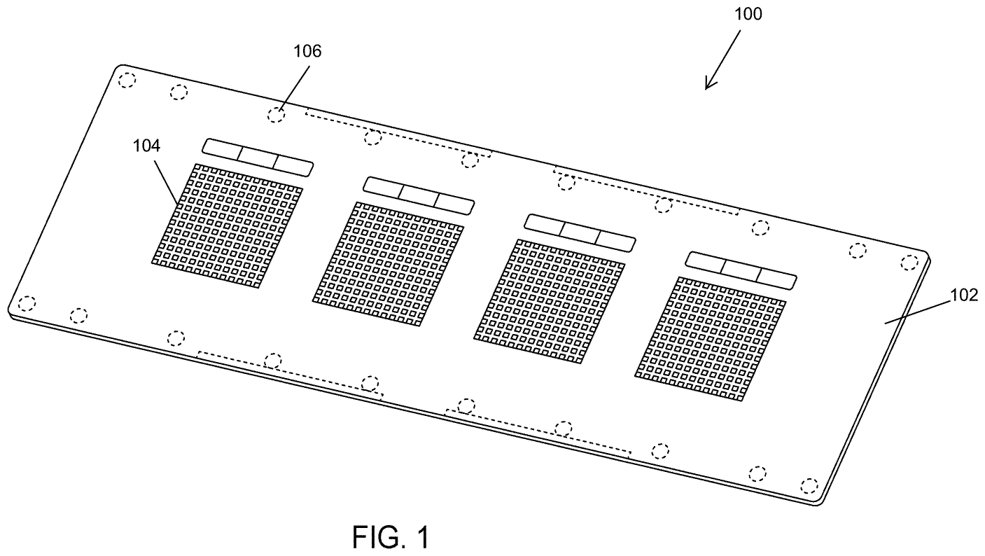

In some embodiments, the high density cell cultivation platform can be a microfabricated device (or a “chip”). As used herein, a microfabricated device or chip may define a high density array of microwells (or experimental units). For example, a microfabricated chip comprising a “high density” of microwells may include about 150 microwells per cm² to about 160,000 microwells or more per cm² (for example, at least 150 microwells per cm², at least 250 microwells per cm², at least 400 microwells per cm², at least 500 microwells per cm², at least 750 microwells per cm², at least 1,000 microwells per cm², at least 2,500 microwells per cm², at least 5,000 microwells per cm², at least 7,500 microwells per cm², at least 10,000 microwells per cm², at least 50,000 microwells per cm², at least 100,000 microwells per cm², or at least 160,000 microwells per cm²). A substrate of a microfabricated chip may include about or more than 10,000,000 microwells or locations. For example, an array of microwells may include at least 96 locations, at least 1,000 locations, at least 5,000 locations, at least 10,000 locations, at least 50,000 locations, at least 100,000 locations, at least 500,000 locations, at least 1,000,000 locations, at least 5,000,000 locations, or at least 10,000,000 locations. The arrays of microwells may form grid patterns, and be grouped into separate areas or sections. The dimensions of a microwell may range from nanoscopic (e.g., a diameter from about 1 to about 100 nanometers) to microscopic. For example, each microwell may have a diameter of about 1 μm to about 800 μm, a diameter of about 25 μm to about 500 μm, or a diameter of about 30 μm to about 100 μm. A microwell may have a diameter of about or less than 1 μm, about or less than 5 μm, about or less than 10 μm, about or less than 25 μm, about or less than 50 μm, about or less than 100 μm, about or less than 200 μm, about or less than 300 μm, about or less than 400 μm, about or less than 500 μm, about or less than 600 μm, about or less than 700 μm, or about or less than 800 μm. In exemplary embodiments, the diameter of the microwells can be about 100 m or smaller, or 50 m or smaller. A microwell may have a depth of about 25 μm to about 100 μm, e.g., about 1 μm, about 5 μm, about 10 μm, about 25 μm, about 50 μm, about 100 μm. It can also have greater depth, e.g., about 200 μm, about 300 μm, about 400 μm, about 500 μm. The microfabricated chip can have two major surfaces: atop surface and a bottom surface, where the microwells have openings at the top surface. Each microwell of the microwells may have an opening or cross section having any shape, e.g., round, hexagonal, square, or other shapes. Each microwell may include sidewalls. For microwells that are not round in their openings or cross sections, the diameter of the microwells described herein refer to the effective diameter of a circular shape having an equivalent area. For example, for a square shaped microwell having side lengths of 10×10 microns, a circle having an equivalent area (100 square microns) has a diameter of 11.3 microns. Each microwell may include a sidewall or sidewalls. The sidewalls may have a cross-sectional profile that is straight, oblique, and/or curved. Each microwell includes a bottom which can be flat, round, or of other shapes. The microfabricated chip (with the microwells thereon) may be manufactured from a polymer, e.g., a cyclic olefin polymer, via precision injection molding or some other process such as embossing. Other material of construction is also available, such as silicon and glass. The chip may have a substantially planar major surface. FIG. 1 shows a schematic depiction of a microfabricated chip, whose edges are generally parallel to the directions of the rows and the columns of the microwells on the chip.

The high density microwells on the microfabricated chip can be used for receiving a sample comprising at least one biological entity (e.g., at least one cell). The term “biological entity” may include, but is not limited to, an organism, a cell, a cell component, a cell product, and a virus, and the term “species” may be used to describe a unit of classification, including, but not limited to, an operational taxonomic unit (OTU), a genotype, a phylotype, a phenotype, an ecotype, a history, a behavior or interaction, a product, a variant, and an evolutionarily significant unit. The high density microwells on the microfabricated chip can be used to conduct various experiments, such as growth or cultivation or screening of various species of bacteria and other microorganisms (or microbes) such as aerobic, anaerobic, and/or facultative aerobic microorganisms. The microwells may be used to conduct experiments with eukaryotic cells such as mammalian cells. Also, the microwells can be used to conduct various genomic or proteomic experiments, and may contain cell products or components, or other chemical or biological substances or entities, such as a cell surface (e.g., a cell membrane or wall), a metabolite, a vitamin, a hormone, a neurotransmitter, an antibody, an amino acid, an enzyme, a protein, a saccharide, ATP, a lipid, a nucleoside, a nucleotide, a nucleic acid (e.g., DNA or RNA), a chemical, e.g., a dye, enzyme substrate, etc.

Fluorescence screening of microwells on a microfabricated chip may involve interrogating microwells by a spectroscopic method, e.g., using a fluorescence detector to detect fluorescence emitted from the microwells, or lack of fluorescence emitted from the microwells, and those microwells that are determined to meet certain criteria (e.g., emitting fluorescence at certain wavelength or not emitting fluorescence at a certain wavelength) can be selected and the contents of at least one microwell or a portion of the contents of at least one microwell transferred to a second location.

In some embodiments, the high density cell cultivation platform can be droplet based, e.g., instead of array(s) of wells as experimental units on a microfabricated chip, a population of discrete droplets can be used to retain cells, media and other components for cell cultivation. Droplet generation methods, especially when combined with cell-sorter-on-a-chip type instrumentation, may be used to grow and screen microbes from a complex environmental sample. Droplets may be produced at several hundred Hz, meaning millions of drops can be produced in a few hours. A simple microfluidic chip-based device may be used to generate droplets and the droplets may be engineered to contain a single cell. A system for generating droplets containing cell suspensions may contain one or small numbers of cells. The droplets can be emulsions, double emulsion, hydrogel, bubbles and complex particles, etc. For example, aqueous drops may be suspended in a nonmiscible liquid keeping them apart from each other and from touching or contaminating any surfaces. The volume of a droplet can be somewhere between 10 fl and 1 μL, and highly monodisperse droplets can be made from a few nanometers up to 500 μm in diameter. A droplet-based microfluidic system may be used to generate, manipulate, and/or incubate small droplets. Cell survival and proliferation can be similar to control experiments in bulk solution. Fluorescence screening of droplets may be done on-chip and at a rate of, for example, 500 drops per second. Droplets may be merged to create a new droplet or a reagent added to a droplet. Droplets can be passed in a microchannel in a single file and interrogated by a spectroscopic method, e.g., using a fluorescence detector to detect fluorescence emitted from the droplets, or lack of fluorescence emitted from the droplets, and those droplets that are determined to meet certain criteria (e.g., emitting fluorescence at certain wavelength or not emitting fluorescence at a certain wavelength) can be selected via diversion into a branched channel from which the droplet can be pooled or harvested. The diversion or switching of flow can be accomplished by valves, pump, applying an external electric field, etc.

In various embodiments, a cell may be Archaea, Bacteria, or Eukaryota (e.g., fungi). For example, a cell may be a microorganism, such as an aerobic, anaerobic, or facultative aerobic microorganisms. A virus may be a bacteriophage. Other cell components/products may include, but are not limited to, proteins, amino acids, enzymes, saccharides, adenosine triphosphate (ATP), lipids, nucleic acids (e.g., DNA and RNA), nucleosides, nucleotides, cell membranes/walls, flagella, fimbriae, organelles, metabolites, vitamins, hormones, neurotransmitters, and antibodies.

For the cultivation of cells, a nutrient is often provided. A nutrient may be defined (e.g., a chemically defined or synthetic medium) or undefined (e.g., a basal or complex medium). A nutrient may include or be a component of a laboratory-formulated and/or a commercially manufactured medium (e.g., a mix of two or more chemicals). A nutrient may include or be a component of a liquid nutrient medium (i.e., a nutrient broth), such as a marine broth, a lysogeny broth (e.g., Luria broth), etc. A nutrient may include or be a component of a liquid medium mixed with agar to form a solid medium and/or a commercially available manufactured agar plate, such as blood agar.

A nutrient may include or be a component of selective media. For example, selective media may be used for the growth of only certain biological entities or only biological entities with certain properties (e.g., antibiotic resistance or synthesis of a certain metabolite). A nutrient may include or be a component of differential media to distinguish one type of biological entity from another type of biological entity or other types of biological entities by using biochemical characteristics in the presence of specific indicator (e.g., neutral red, phenol red, eosin y, or methylene blue).

A nutrient may include or be a component of an extract of or media derived from a natural environment. For example, a nutrient may be derived from an environment natural to a particular type of biological entity, a different environment, or a plurality of environments. The environment may include, but is not limited to, one or more of a biological tissue (e.g., connective, muscle, nervous, epithelial, plant epidermis, vascular, ground, etc.), a biological fluid or other biological product (e.g., amniotic fluid, bile, blood, cerebrospinal fluid, cerumen, exudate, fecal matter, gastric fluid, interstitial fluid, intracellular fluid, lymphatic fluid, milk, mucus, rumen content, saliva, sebum, semen, sweat, urine, vaginal secretion, vomit, etc.), a microbial suspension, air (including, e.g., different gas contents), supercritical carbon dioxide, soil (including, e.g., minerals, organic matter, gases, liquids, organisms, etc.), sediment (e.g., agricultural, marine, etc.), living organic matter (e.g., plants, insects, other small organisms and microorganisms), dead organic matter, forage (e.g., grasses, legumes, silage, crop residue, etc.), a mineral, oil or oil products (e.g., animal, vegetable, petrochemical), water (e.g., naturally-sourced freshwater, drinking water, seawater, etc.), and/or sewage (e.g., sanitary, commercial, industrial, and/or agricultural wastewater and surface runoff).

FIG. 1 is a perspective view illustrating a microfabricated device or chip in accordance with some embodiments. Chip 100 includes a substrate shaped in a microscope slide format with injection-molded features on top surface 102. The features include four separate microwell arrays (or microarrays) 104 as well as ejector marks 106. The microwells in each microarray are arranged in a grid pattern with well-free margins around the edges of chip 100 and between microarrays 104.

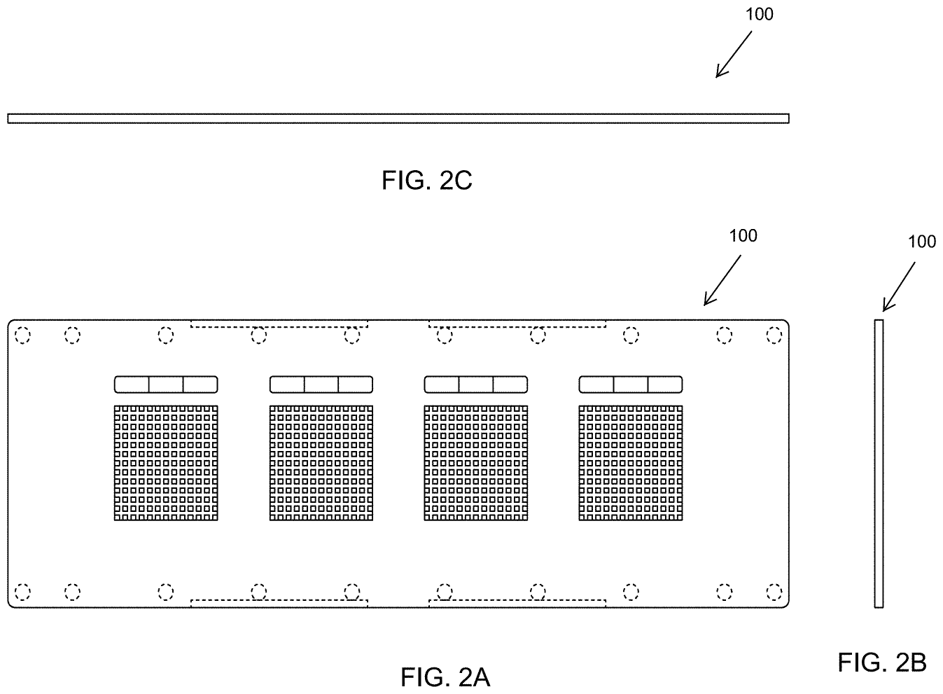

FIGS. 2A-2C are top, side, and end views, respectively, illustrating dimensions of chip 100 in accordance with some embodiments. In FIG. 2A, the top of chip 100 is approximately 25.5 mm by 75.5 mm. In FIG. 2B, the end of chip 100 is approximately 25.5 mm by 0.8 mm. In FIG. 2C, the side of chip 100 is approximately 75.5 mm by 0.8 mm.

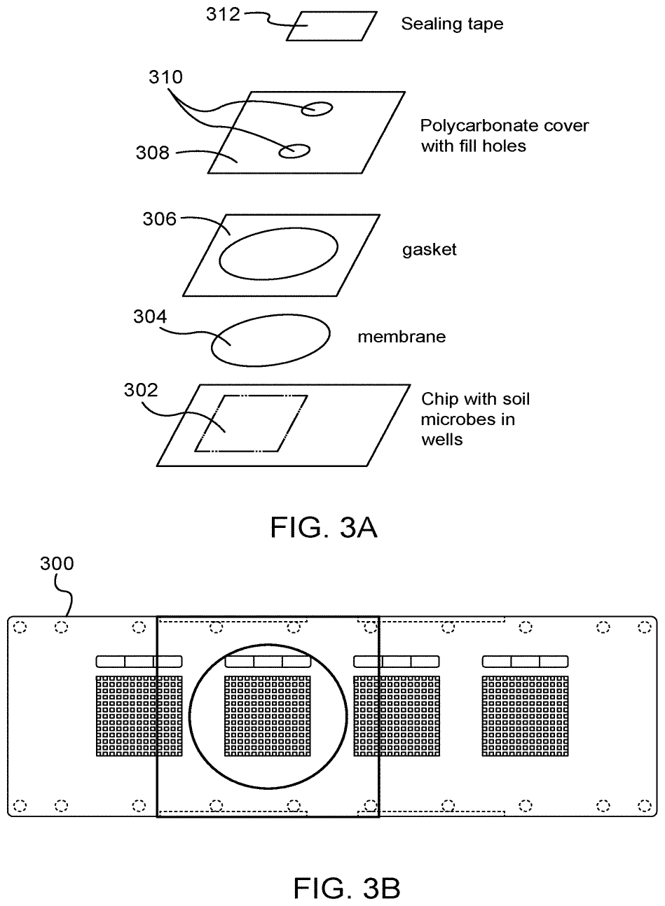

After a sample is loaded on a microfabricated device, a membrane may be applied to at least a portion of a microfabricated device. FIG. 3A is an exploded diagram of the microfabricated device 300 shown from a top view in FIG. 3B in accordance with some embodiments. Device 300 includes a chip with an array of wells 302 holding, for example, soil microbes. A membrane 304 is placed on top of the array of wells 302. A gasket 306 is placed on top of the membrane 304. A cover 308 with fill holes 310 is placed on top of the gasket 306. Finally, sealing tape 312 is applied to the cover 308.

A membrane may cover at least a portion of a microfabricated device including one or more experimental units or microwells. For example, after a sample is loaded on a microfabricated device, at least one membrane may be applied to at least one microwell of a high density array of microwells. A plurality of membranes may be applied to a plurality of portions of a microfabricated device. For example, separate membranes may be applied to separate subsections of a high density array of microwells.

A membrane may be connected, attached, partially attached, affixed, sealed, and/or partially sealed to a microfabricated device to retain at least one biological entity in the at least one microwell of the high density array of microwells. For example, a membrane may be reversibly affixed to a microfabricated device using lamination. A membrane may be punctured, peeled back, detached, partially detached, removed, and/or partially removed to access at least one biological entity in the at least one microwell of the high density array of microwells.

A portion of the population of cells in at least one experimental unit, well, or microwell may attach to a membrane (via, e.g., adsorption). If so, the population of cells in at least one experimental unit, well, or microwell may be sampled by peeling back the membrane such that the portion of the population of cells in the at least one experimental unit, well, or microwell remains attached to the membrane.

In some embodiments, the population of cells in at least one experimental unit, well, or microwell may be sampled by puncturing the membrane with a sampling device such as a pin or an aspiration device and transferring a portion of the population of cells in the at least one experimental unit to a target location.

A membrane may be impermeable, semi-permeable, selectively permeable, differentially permeable, and/or partially permeable to allow diffusion of at least one nutrient into the at least one microwell of a high density array of microwells. For example, a membrane may include a natural material and/or a synthetic material. A membrane may include a hydrogel layer and/or filter paper. In some embodiments, a membrane is selected with a pore size small enough to retain at least some or all of the cells in a microwell. For mammalian cells, the pore size may be a few microns and still retain the cells. However, in some embodiments, the pore size may be less than or equal to about 0.2 μm, such as 0.1 μm. An impermeable membrane has a pore size approaching zero. It is understood that the membrane may have a complex structure that may or may not have defined pore sizes.

In one aspect, the present invention provides a method for assessing a status, e.g., metabolic activity, of at least one cell in a pure or mixed cell culture in an anaerobic condition or atmosphere. The cell culture may be loaded in a traditional cell culture platform such as petri dishes or compartments of 96-well plates, 384-well plates. Or they may be loaded in one or more experimental units of a high density cell cultivation platform as described herein. The cell culture platform may be maintained in an anaerobic chamber supplied with carbon dioxide and hydrogen required for cell metabolic activities in anaerobic conditions. The cell culture may be covered by a membrane permeable to such gases. The conversion of resorufin in the monitored area (e.g., the wells of the plate, or microwells on a microfabricated chip, with or without having to peel off the membrane) can be monitored by the intensity of fluorescence given off by resorufin, with a resolution sufficient to distinguish between different cell loading locations in the culture platform. The measurement result can indicate the level of metabolic activity of the cell or cells in the cell culture.

As used herein, metabolic activity of cells includes cell activity in cell growth, cell division, and proliferation. The at least one cell can include a prokaryotic cell, a eukaryotic cell, a bacterial cell, etc.

In some embodiments, based on the measured fluorescence of resorufin, it is determined whether at least one biological entity is present in the cell culture. For example, based on the measured fluorescence, one can determine whether a species of anaerobic bacteria is present. In some embodiments, based on the measurement result, some cells from the cell culture can be selected/picked and transferred to a target location.

In an anaerobic condition, resazurin can be easily reduced by the cell culture media or environment, and is therefore unsuitable for use as an indicator of metabolic activity of the cells. In such environment, the resorufin=>dihyrdoresorufin reaction takes more reduction potential, and the reduction of resorufin to dihydroresorufin can be used in a manner that is analogous to the more common resazurin=>resorufin for detecting cell metabolic activity. Preferably, when using resorufin in such a setting, the cells can be kept at a reduction potential above that of resorufin, but still low enough to remove oxygen and keep the cells viable. Type of media, PH, and reagents or other species in the culture media can affect the reduction potential of the cells and that of resorufin.

Resorufin can be obtained in powder form. It can then be introduced to a cell culture medium in an anaerobic atmosphere, e.g., an anaerobic chamber. Any oxygen remaining in the cell culture can be drained by vacuum/flushing the anaerobic chamber with CO₂, N₂ and/or other gases. The medium loaded with resorufin can then be loaded in a culture platform subject to fluorescence monitoring.

In other embodiments, a method of using a high density cell cultivation platform including a plurality of experimental units is provided, which includes: loading a sample onto the platform such that at least one experimental unit includes at least one cell, an amount of a nutrient (or culture media), and resorufin; culturing a plurality of cells from the at least one cell in the at least one experimental unit in an anaerobic atmosphere; and measuring fluorescence of the contents of the at least one experimental unit. The measurement can be done in multiple time points during the course of the culture process after adding resorufin for a predetermined duration of time, or until the difference between the last two measurements is within a preset tolerance.

In some embodiments, the high density cell cultivation platform is a microfabricated device having a top surface defining an array of microwells as experimental units, the microwells having a surface density of at least 500 microwells per cm² or at least 750 microwells per cm². In some embodiments, the microwells may each have a volume of no more than 5 nL.

In some embodiments, based on the measured fluorescence, it is determined whether at least one biological entity in present in the at least one microwell. In some embodiments, based on the measured fluorescence, the method further comprises selecting some cells (one or more cells) from the at least one experimental unit (e.g., microwell) and transferring the selected at least one cell to a target location (e.g., another microwell on the same chip, a destination cell culture compartment or a well in a 96-well plate, etc.).

### Example 1

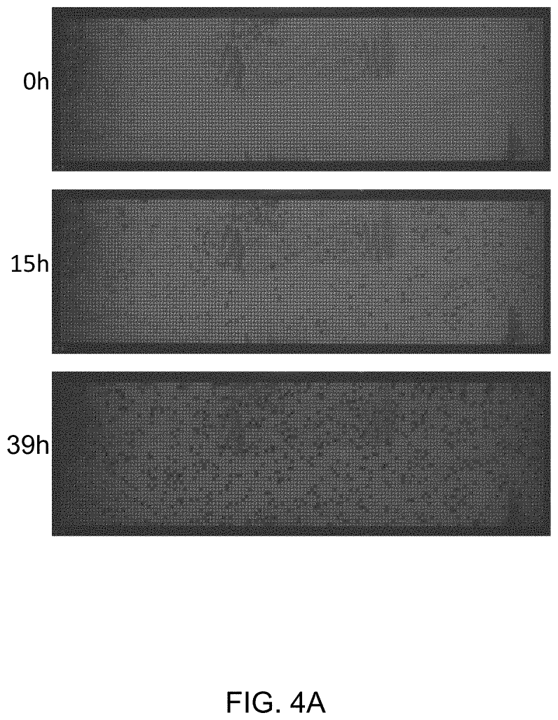

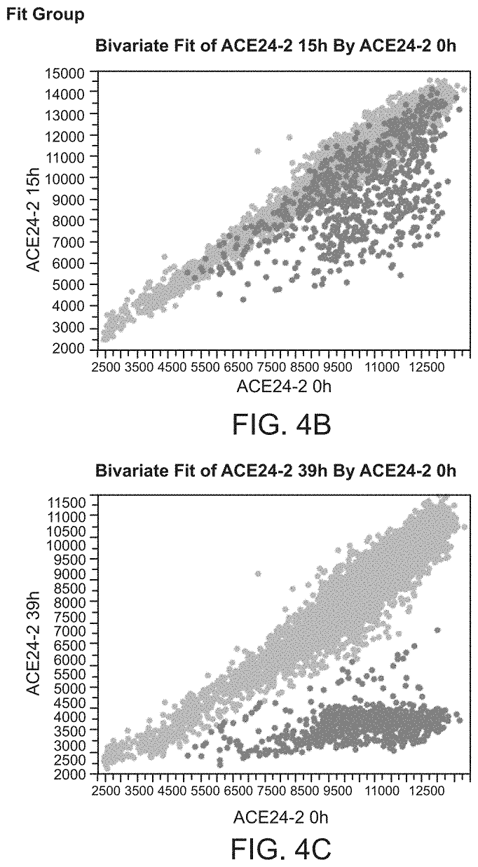

In this example, Bacteroides fragilis growth on microwells of a microfabricated chip in anaerobic condition was monitored with resorufin indicator. FIG. 4A shows the microfabricated chip at 0, 15, and 39 hours of cultivation (fluorescence excited at 532 nm), where growth of bacteria in individual wells is signified as a decrease in fluorescence intensity (or brightness); FIG. 4B shows signal intensities at 0 hours vs 15 hours, and FIG. 4C show signal intensities at 0 hours vs 39 hours, where blue (darker) dots representing microwells with decreased signal are “positives” and contain the bacteria, and those in gray (lighter shaded dots) are “negatives” and are empty. The results were further confirmed by transferring the bacterial cells from the microwells into a 96-well plate, in which negatives remain negative, and positives result in further growth (indicated as cloudiness of the media).

### Example 2

Human gut microbiome (HGM) sample was processed and analyzed in this example. More specifically, clonal populations from human fecal samples were cultured anaerobically on microfabricated chips, many isolates that were present in less than 1% of the microbial population were recovered.

One key aspect of assembling isolate libraries is ensuring that they are representative of the microbial communities from which they are derived. Rare and/or slow-growing species can be missed during cultivation using petri dishes. Although they may comprise only a small percentage of the overall mix of microbes, rare and/or slow-growing species can be key players in maintaining the overall equilibrium of a community and may even be keystone species. Rare species can be missed if they are not well adapted to the medium being used, or if they inherently grow slowly, or if they are outcompeted by strains that are abundant and grow quickly.

In this example, appropriately diluted complex sample are diluted and distributed to microwells of a microfabricated device (or array) so that each microwell contains only a single bacterium, therefore there will be no direct growth competition between strains. Second, because of the ease and speed with which multiple parallel experiments can be conducted in the high-density array platform, it is practical to increase library diversity by strategically testing several different media.

The microfabricated chip and the manipulation thereof was completely in an anaerobic chamber such that all steps—from array loading and sealing, incubation of the arrays, imaging of arrays for monitoring culture growth, transfer and sealing of metabolically active cultures in 96-well plates for scale up, and the incubation of these 96-well plates—can all be accomplished without samples ever leaving the chamber and without needing to manage dozens or hundreds of Petri dishes.

Resorufin was used as an indicator of anaerobic metabolism for the HGM sample. The biological reduction of resorufin to dihydroresorufin by metabolic byproducts of anaerobic fermentation occurs more rapidly than does the abiotic reduction of resorufin by H₂, allowing empty wells to be discriminated from those containing cultures. This discrimination under anaerobic conditions is accomplished by looking at the change in signal for each well from time zero to any later timepoint. Wells showing a greater decrease in fluorescence than the abiotic background signal change shown by empty wells are designated culture positive.

### Methods

Healthy human fecal samples (The BioCollective, Colorado) for which there was paired metagenomic data (CosmosID, Maryland) were cultured. Preliminary experiments on all samples were performed to determine appropriate cell densities for loading onto the array of microwells of microfabricated chip in order to achieve an optimal Poisson distribution, i.e., a maximum number of wells containing a single bacterium with a minimum number of wells containing more than one bacterium. A target of 0.3 cells per well typically results in an optimal number of singly occupied wells.

Arrays and all plastic consumables and equipment were acclimated to anaerobic conditions by degassing overnight within an anaerobic chamber. All parts of the loading apparatus were sterilized by autoclaving before use. Cell dilutions were prepared in the various media to be tested, mixed with the growth indicator resorufin to a final concentration of 50 μM, and volumes of 3.0 mL were loaded onto each array inside the anaerobic chamber, then sealed. Arrays were next scanned to provide a time-zero reading of green fluorescence from resorufin and incubated anaerobically for 16 to 65 hours at 37° C. with daily imaging to identify wells containing active cultures. Aliquots from the subset of wells showing positive growth were transferred from the arrays into 96-well plates and incubated anaerobically for 5 to 10 days. Isolates were identified with partial 16S RNA Sanger sequencing; those with insufficient resolution at the taxa level were removed from further analysis.

The media evaluated in this Example were Gifu anaerobic medium (GAM), brain heart infusion (BHI), brucella blood agar (BRU), peptone yeast-extract glucose broth (PYGB), and yeast casitone fatty acids agar with carbohydrates (YCFAC).

### Results

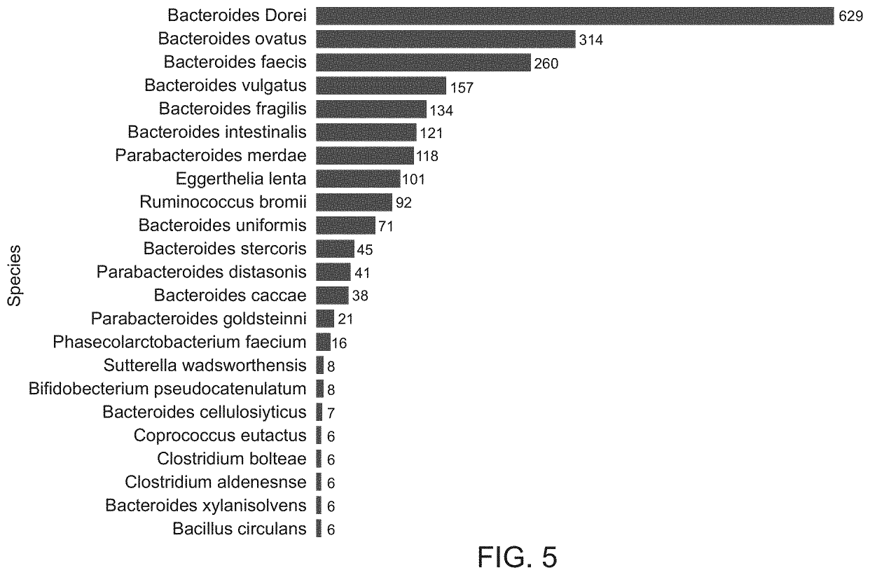

Six unique human fecal samples from healthy donors for which paired metagenomic data was available were cultured and yielded 2790 isolates. Sanger sequencing of isolates indicated 5 phyla, 9 classes, 12 orders, 19 families, 27 genera, and 45 species; 23 species for which at least six isolates were recovered are shown in a bar graph (FIG. 5 shows isolates at species level recovered from six healthy-donor fecal samples).

An additional 22 species were identified in the six samples for which there were five or fewer isolates (see Table 1 below).

TABLE 1
Species for which ≤5 isolates recovered

| Tax N < 5 | |
|---|---|
| N = 1 | N = 2 |
| Akkermansia | Alistipes |
| muciniphila | indistinctus |
| Alistipes | Bacteroides |
| onderdonkii | massiliensis |
| Bilophila | Bifidobacterium |
| wadsworthia | longum |
| Blautia | Clostridium |
| coccoides | hylemonae |
| Christensenella | Escherichia/Shigella |
| minuta | fergusonii |
| Clostridium | Gordonibacter |
| scindens | pamelaeae |
| Coprococcus comes | Paraprevotella chara |
| Dorea longicatena | N =3 |
| Gordonibacter | Clostridium |
| faecihominis | citroniae |
| Lactonifactor | Eggerthella |
| longoviformis | sinensis |
| | Hungatella effluvii |
| | Lactobacillus |
| | paracasei |
| | N = 5 |
| | Enterococcus durans |

Not only were these isolates relatively rare in terms of their capture in culture, but in nearly every case, paired metagenomics data had predicted a prevalence of <0.2% for these species or genera to be found in the samples.

Comparison of Sanger 16S RNA sequencing data for identification at the species level with predicted prevalence from metagenomic data highlights the strength of the microfabricated chip-based high density cultivation platform in capturing rare members of a microbial community. The 33 species isolated from sample HGM-6 represent five phyla, eight classes, 10 orders, 16 families, and 20 genera (see Table 2: Taxa at species level for recovered isolates from a single sample (HGM-6) with percent relative abundance by Metagenomics (MTG)). Of these 33 species, 18 were predicted to be present at <1.0% abundance, demonstrating the ability of the system to isolate rare strains. Seven species not identified in the same sample by metagenomic analysis were also identified by the methodology based on the high density platform.

TABLE 2

| PHYLUM | CLASS | ORDER | FAMILY | GENUS | SPECIES | # | % MTG |
|---|---|---|---|---|---|---|---|
| Actinobacteria | Actinobacteria | Bifidobacteriales | Bifidobacteriaceae | Bifidobacterium | Bifidobacterium longum | 2 | 0.88% |
| | | Coriobacteriales | Coriobacteriaceae | Eggerthella | Eggerthella lenta | 6 | 0.03% |
| | | | | Gordonibacter | Gordonibacter faecihominis | 1 | N/A |
| Bacteroidetes | Bacteroidia | Bacteroidales | Bacteroidaceae | Bacteroides | Bacteroides caccae | 38 | 0.68% |
| | | | | | Bacteroides dorei | 629 | 6.15% |
| | | | | | Bacteroides faecis | 182 | 0.01% |
| | | | | | Bacteroides fragilis | 54 | 0.13% |
| | | | | | Bacteroides intestinalis | 121 | 4.86% |
| | | | | | Bacteroides ovatus | 110 | 1.31% |
| | | | | | Bacteroides uniformis | 66 | 2.88% |
| | | | | | Bacteroides vulgatus | 30 | 3.89% |
| | | | | | Bacteroides xylanisolvens | 5 | 0.40% |
| | | | Porphyromonadaceae | Parabacteroides | Parabacteroides distasonis | 39 | 0.62% |
| | | | | | Parabacteroides goldsteinii | 21 | 0.05% |
| | | | | | Parabacteroides merdae | 118 | 2.54% |
| | | | Prevotellaceae | Paraprevotella | Paraprevotella clara | 2 | 0.41% |
| | | | Rikenellaceae | Alistipes | Alistipes indistinctus | 2 | <0.01% |
| | | | | | Alistipes onderdonkii | 1 | 0.25% |
| Firmicutes | Bacilli | Bacillales | Bacillaceae_1 | Bacillus | Bacillus circulans | 6 | N/A |
| | | Lactobacillales | Enterococcaceae | Enterococcus | Enterococcus durans | 5 | N/A |
| | | | Lactobacillaceae | Lactobacillus | Lactobacillus paracasei | 2 | N/A |
| | Clostridia | Clostridiales | Christensenellaceae | Christensenella | Christensenella minuta | 1 | 0.14% |
| | | | Lachnospiraceae | Clostridium_{—}X1Va | Clostridium aldenense | 6 | N/A |
| | | | | | Clostridium bolteae | 1 | 0.02% |
| | | | | Coprococcus | Coprococcus comes | 1 | 1.16% |
| | | | | | Coprococcus eutactus | 6 | 0.36% |
| | | | | Dorea | Dorea longicatena | 1 | 0.76% |
| | | | | Lactonifactor | Lactonifactor longoviformis | 1 | N/A |
| | | | Ruminococcaceae | Ruminococcus | Ruminococcus bromii | 1 | 3.09% |
| | Negativicutes | Selenomonadales | Acidaminococcaceae | Phascolarctobacte | Phascolarctobacterium | 16 | N/A |
| Proteobacteria | Beta- | Burkholderiales | Sutterellaceae | Sutterella | Sutterella wadsworthensis | 8 | 0.39% |
| | proteobacteria | | | | | | |
| | Delta- | Desulfo- | Desulfovibrionaceae | Bilophila | Bilophila wadsworthia | 1 | 0.07% |
| | proteobacteri | vibrionales | | | | | |
| Verruco- | Verrucomicrobiae | Verrucomicrobiales | Verrucomicrobiaceae | Akkermansia | Akkermansia muciniphila | 1 | 0.02% |
| microbia | | | | | | | |

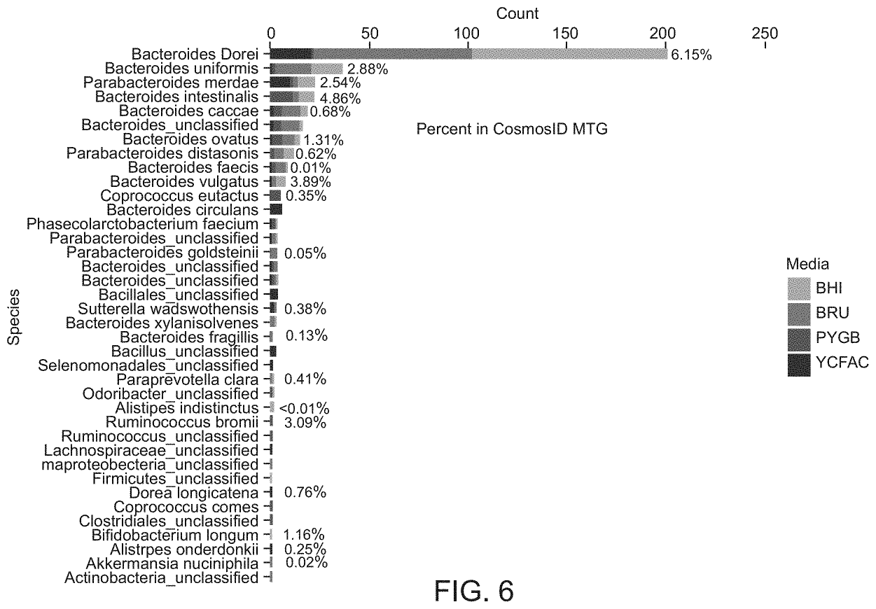

In addition, multiple media were evaluated in parallel to determine the presence of difficult-to-culture strains that may be present in a sample. See FIG. 6 which shows relative population of strains captured at genus level by different media.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method for identifying a status of at least one cell in a cell culture including resorufin in an anaerobic atmosphere, comprising:
while maintaining the cell culture in an anaerobic atmosphere, measuring the extent of reduction of resorufin in the cell culture to dihyrdoresorufin.

2. The method of claim 1, wherein the measuring comprises measuring the fluorescence of the cell culture.

3. The method of claim 1, wherein the at least one cell is loaded in one or more microwells of a microfabricated chip.

4. The method of claim 1, wherein the at least one cell is loaded in one or more droplets on a droplet-based platform.

5. The method of claim 1, wherein the at least one cell comprises a prokaryotic cell,

6. The method of claim 1, wherein the at least one cell comprises a eukaryotic cell.

7. The method of claim 1, wherein the at least one cell comprises a bacterial cell.

8. The method of claim 1, wherein the measuring is performed for a plurality of times.

9. The method of claim 1, wherein cell culture is prepared by first mixing resorufin with a culture media, and then combining the at least one cell with the resorufin-loaded culture media.

10. The method of claim 1, wherein the status of the at least one cell comprises the metabolic activity of the at least one cell.

11. The method of claim 1, further comprising: determining the presence or absence of at least one biological entity in the cell culture based on the measured extent of reduction of resorufin to dihydroresorufin.

12. A method of using a high density cell cultivation platform comprising a plurality of experimental units, comprising:
loading a sample onto the high density cell cultivation platform such that at least one experimental unit of the plurality of experimental units includes at least one cell, an amount of a nutrient, and resorufin;
culturing a plurality of cells from the at least one cell in the at least one experimental unit in an anaerobic atmosphere; and
measuring fluorescence of the contents of the at least one experimental unit.

13. The method of claim 12, wherein the high density cell cultivation platform is a microfabricated device having a top surface defining an array of microwells as experimental units, the microwells having a surface density of at least 500 microwells per cm² or at least 750 microwells per cm².

14. The method of claim 13, wherein the microwells each have a volume of no more than 5 nL.

15. The method of claim 12, further comprising: determining the presence or absence of at least one biological entity in the at least one experimental unit based on the measured fluorescence.

16. The method of claim 12, further comprising: based on the measured fluorescence, determining whether to select and transfer some cells from the plurality of cells to one or more target location.

17. The method of claim 12, wherein the high density cell cultivation platform is a droplet-based platform and the plurality of experimental units each comprise to a droplet on the droplet-based platform.
